(19) United States
(12) Patent Application Publication
(10) Pub. No.: US 2007/0213655 A1
(43) Pub. Date: Sep. 13, 2007

(54) Allograft or Autograft Inserter

(76) Inventor: Chad J. Prusmack, Denver, CO (US)

Correspondence Address: GLOBUS MEDICAL, INC.;ATTN: BRIAN MALM, VALLEY FORGE BUSINESS CENTER, 2560 GENERAL ARMISTEAD AVENUE, AUDUBON, PA, 19403, US

(21) Appl. No.: 11/683,385

(22) Filed: Mar. 7, 2007

Related U.S. Application Data
(60) Provisional application No. 60/779,947, filed on Mar. 7, 2006.

(51) Int. Cl. A61M 31/00

(52) U.S. Cl. 604/57

(57) ABSTRACT

An inserter device for autograft or allograft bone is disclosed.

### CROSS-REFERENCE TO RELATED APPLICATIONS

The present application claims priority to U.S. Provisional Application No. 60/779,947, filed Mar. 7, 2006, the entire contents of which are incorporated herein by reference.

### BACKGROUND OF THE INVENTION

In spine fusion surgery, a goal of the surgery is to have two adjacent vertebrae “fused together” with particular types of bone products, which will cause one vertebra to fuse to the other. A common type of fusion technique is done in the interdiscal space, which is called an interbody fusion. This technique fuses adjacent vertebrae. What this technique involves is to remove the intervening disc material allowing only the adjacent ‘bony’ vertebra to be effacing an area without any disc material. The interbody technique then commonly involves either placing cages which hold fusion material, either the patient's own bone or processed bone from a bone bank. To augment interbody fusions with grafts in cages, directly placing either bone type directly into the interbody space is a common technique. This can sometimes also be used as a substitute for a cage. When one uses minimally invasive techniques to do an interbody spine fusion, the space available-to introduce either the cage or allograft is reduced to about a 2 mm diameter. Therefore manipulating loose bone graft can commonly be messy, inaccurate and difficult through a minimally invasive port.

Therefore, it would be advantageous to have a particular tool that could easily and safely introduce bone graft into the freshly evacuated disc space to either augment or substitute for an interbody implantation.

### BRIEF DESCRIPTION OF THE DRAWINGS

The invention will be more readily understood with reference to the embodiments thereof illustrated in the attached figures, in which:

FIG. 1 is an exploded view of one embodiment of a minimally invasive bone graft inserter according to the invention;

FIG. 2 is an end view of the device of FIG. 1;

FIG. 3 is a perspective view of another embodiment of a bone graft inserter according to the invention; and

FIG. 4 is a schematic view of one application of the device of FIGS. 1 and 3.

### DETAILED DESCRIPTION OF THE INVENTION

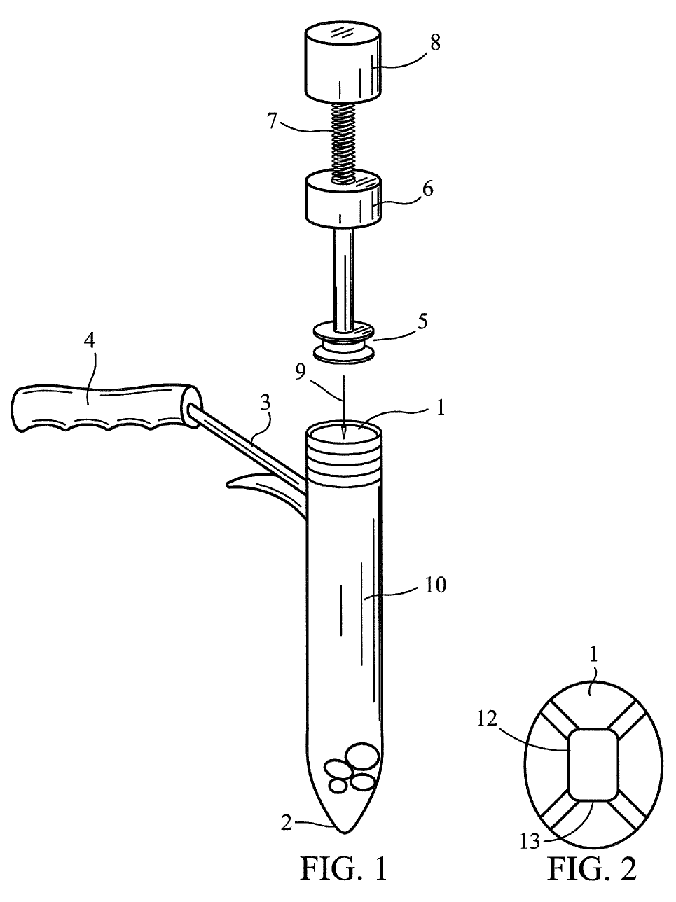

Referring first to FIG. 1, one embodiment of a minimally invasive allograft or autograft inserter 10 according to the invention is shown. The illustrated embodiment possesses some beneficial features, which are described in greater detail below. First, inserter 10 may have a bayoneted handle 3, 4. This is used in order to keep a user's hand out of the way while manipulating the inserter down a small cannula. Inserter 10 can be made out of titanium and mayor may not be radiolucent. In one embodiment, the tip 2 may be tapered and smooth, such that it will not harm the nerve root or bony structures deep in the operative field. Bone in the form of allograft or autograft may be inserted in the orifice 1 and packed down deep towards the orifice, which would be the “bone delivery” side 2. According to one embodiment, the bone could be crushed or have already been through a bone mill prior to use.

After the bone graft has been already inserted into inserter 10, a second assembly is the screw and top (5,6,7,8) which would be placed on inserter 10 as depicted by arrow 9, and screwed into place with the top 6. In one variation, there may be at least two types of ways to advance and squeeze out the allograft. A first method utilizes a screwing device labeled 7 which would work by turning the top portion 8 clockwise, which would cause threaded portion 7 to rotate and advance or push deeper through the screw mechanism 6 causing the plunger 5 to move deeper in inserter 10 such that top portion 8 may be fully screwed down or seated and plunger 5 would be adjacent the orifice 2, and therefore the bone would be introduced or expelled out of orifice 2. According to another embodiment, another type of inserter would have a distal portion 7 as smooth and a hammer could be used to slowly tap top portion 8, and this would cause the plunger to move downward and expel the bone graft through orifice 2.

The actual tip seen in FIG. 2 may have two dimensions. A longer dimension 12, and a shorter dimension 13. This would help with orienting the end of the tube relative to the nerve root and disk space so as not to injure it yet to get the surface area over the disc space for placement of graft. As seen in FIG. 2, the tip 1 may be tapered.

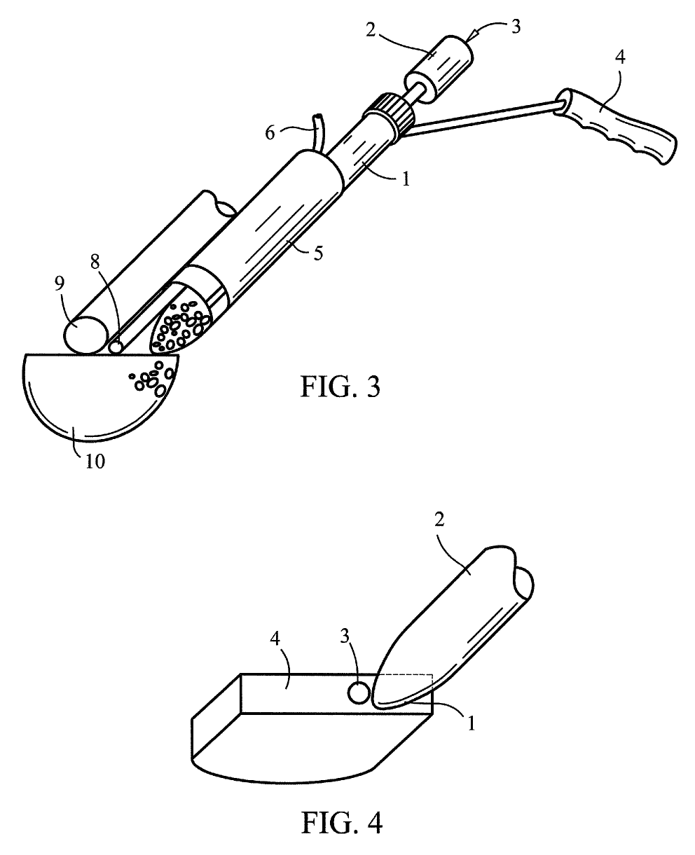

The use of the inserter is depicted in FIG. 3. The disc space is pictured labeled 10. The spine's thecal sac is labeled 9. The adjacent nerve root is labeled as item 8. According to one method of minimally invasive spine surgery, a minimally invasive access port 5 is placed for the entire surgery. Access port 5 retracts the fascia and muscle and is the area where one operates through. In this example, item 6 depicts a retractor nerve root which is retracting the nerve 8 medially away from the area where the allograft will be introduced. The allograft is expelled from the inserter through the orifice 12 and is illustrated going into the disc space progressively as in number 7 and, preferably, not on the nerve 8 or in the epidural space 13.

Referring to FIG. 3, the mechanism is first introduced into the inserter 1. Then number 2 is either screwed or tapped with a hammer such as seen in the arrow 3. This moves the plunger through number 1 down into the distal end labeled 12, and as number 2 is screwed or hammered more, it expels more safely into the disc space and, preferably, not into the nerve root and not into the epidural space labeled 13.

A schematic perspective view of the disc space is provided in FIG. 4. One can see by placing the different diameters 1, that one could either align it with the disc space if you had enough room to do that, or one could turn it and have number 1 adjacent to the nerve root 3, if one did not have as much disc space. In general what this would achieve would be to be able to place bone through a small minimally invasive access cannula safely and preferably without exposing the nerve root to any increased risk of injury, would be able to augment a fusion with more bone graft in the disc space, and would be less messy as to not introduce bone in areas unwanted such as the epidural space, muscle, nerve, or other areas. This would be able to be done much more quickly with this particular utensil and with much better visualization.

What is claimed is:

1. A bone inserter device, comprising:
a cannulated body extending along a longitudinal axis from a proximal end to a distal end and defining a central cannula for housing bone portions, the cannula having a distal orifice adjacent the distal end;
a plunger insertable into the cannulated body and rotatably advanceable along the longitudinal axis to expel the bone portions from the distal orifice; and
wherein the bone portions comprise autograft or allograft bone.
